(19) United States
(12) Patent Application Publication
(10) Pub. No.: US 2014/0287423 A1
(43) Pub. Date: Sep. 25, 2014

(54) DEVICE AND METHOD FOR APPORTIONMENT AND MANIPULATION OF SAMPLE VOLUMES

(71) Applicant: LIFE TECHNOLOGIES CORPORATON, Carlsbad, CA (US)

(72) Inventor: James Nurse, Carlsbad, CA (US)

(21) Appl. No.: 14/354,878

(22) PCT Filed: Oct. 25, 2012
(86) PCT No.: PCT/US2012/061858

§ 371 Date: Apr. 28, 2014

Related U.S. Application Data
(60) Provisional application No. 61/552,741, filed on Oct. 28, 2011.

(51) Int. Cl. C12Q 1/68

(52) U.S. Cl. CPC C12Q 1/6806
USPC 435/6.12; 435/309.1

(57) ABSTRACT

The present invention relates to methods and apparatus for apportionment and manipulation of sample volumes into smaller discrete volumes. The method exploits the interplay of hydrophilic and hydrophobic forces to partition sample volumes. These compartmentalized volumes allow for isolation of samples and partitioning into a localized array that can subsequently be manipulated and analyzed. The partition into extremely small volumes along with the device's inherent portability render our invention versatile for use in many areas, including but not limited to PCR, digital PCR, biological assays for diagnostics and prognostics, cancer diagnosis and prognosis, high throughput screening, single molecule and single cell reactions or assays, the study crystallization and other statistical processes, protein crystallization, drug screening, environmental testing, and the coupling to a wide range of analytical detection techniques for biomedical assays and measurements.

### FIELD

The invention relates to methods and devices for partitioning and manipulating sample volumes into smaller discrete volumes suitable for subsequent biological and chemical assays.

### BACKGROUND

A first step in many chemical and biological applications is the partition of sample volumes into smaller individual volumes for subsequent assays and analysis, where the assay or analysis on each volume may be of the same type or different types. For example, partitioning a sample into smaller volumes is particularly useful in detecting and quantifying nucleic acid molecules using digital PCR. Digital PCR is a technique that allows amplification of a single DNA template from a minimally diluted sample, thus, generating amplicons that are exclusively derived from one template and can be detected with different fluorophores or sequencing to discriminate different alleles (e.g., wild type vs. mutant or paternal vs. maternal alleles).

Several methods exist for partitioning samples. Traditional methods of generating small volumes of sample include the use of a nebulizer to create an aerosol and the mixing of a sample with an immiscible phase to create an emulsion. Other methods for partitioning samples include dispersing into wells or microwells, using self assembled monolayers (SAMs), electrowetting and using microfluidics.

However, none of these methods are ideal and suffer from certain drawbacks. For example, the use of valves and pumps require complex fluidic control and fabricated devices that tend to be expensive, especially if many discrete volumes are involved. Additionally, such flow methods require accurate control of flow rates, which also increases the complexity and expense of the final device or instrument. Accordingly, there is a need in the art for novel approaches for the manipulation of sample volumes.

### SUMMARY

The technology is based on the principle that sample volumes can be partitioned into discrete smaller volumes with only minimal manipulation on the part of an operator. The method of partitioning employs devices that have selectively patterned hydrophilic and hydrophobic regions contained within a cavity or region or area. The partition of the discrete volumes along with its inherent portability further expand upon the versatility for use in many areas, including but not limited to PCR, digital PCR, genotyping, single-cell gene expression analysis, determining copy number variations, biological assays for diagnostics and prognostics, cancer diagnosis and prognosis, DNA methylation assays, high throughput screening, single molecule and single cell reactions or assays, the study crystallization and statistical processes, protein crystallization, drug screening, environmental testing, and the coupling to a wide range of analytical detection techniques for biomedical assays and measurements. The design of the device allows for combinations of manipulation and detection methods to be used in parallel or in series, generating an avenue for complementary detection techniques to be incorporated. Provided herein is a method for partitioning a sample comprising: a) providing a device comprising a surface, enclosed within a cavity, wherein the surface is selectively partitioned into hydrophobic and/or hydrophilic regions; b) filling the device cavity with a liquid that is immiscible with the starting sample; and c) contacting the starting sample with the device surface such that the sample volume is partitioned in an array defined by the hydrophilic and/or hydrophobic regions across the surface. The surface may be hydrophobic such that the selectively partitioned hydrophobic and/or hydrophilic regions are formed by coating hydrophilic spots on said surface. In some embodiments, the hydrophobic surface is a glass surface, or a black anodized aluminum surface with a thin oxide deposition layer. In some embodiments, the surface is hydrophilic such that the selectively partitioned hydrophobic and/or hydrophilic regions are formed by coating hydrophobic spots on said surface. In some embodiments, the hydrophilic and/or hydrophobic regions are further coated with a binding site for biomolecules selected from a group comprising protein molecules, carbohydrate molecules, nucleic acids and fatty acids. In some embodiments, the surface is selectively partitioned in an array of hydrophilic and/or hydrophobic regions. In some embodiments, the surface is selectively partitioned in hydrophilic and/or hydrophobic regions having dimensions of 5-200 μm and may be selectively partitioned in hydrophilic and/or hydrophobic regions using microfabrication techniques. The microfabrication techniques may include depositions, plasmas, masking steps, transfer printing, screen printing, spotting, spin coating with a lift off (lithography) step, vapor deposition with selective marking, vapor deposition with a lift off (parylene deposition) and pin spotting (dip pen nanolithography) or transfer printing. In some embodiments, the device cavity may be formed by adding a dam structure along the margins of the patterned surface and attaching a cover to the top of the dam. In some embodiments, the cover is a glass lid. In some embodiments, the device cavity has one or more fill ports for loading the starting sample volume into the device cavity which may or may not be included in the cover. In some embodiments, the immiscible liquid is an organic liquid, such as for example a mineral oil such as silicon oil or a fluorinated oil. In some embodiments, the starting sample is partitioned into smaller volumes by contacting it with the selectively partitioned surface by oscillating the device in a to and fro motion or by contacting the surface with the selectively partitioned surface by employing a magnetic force across the surface. In some embodiments, the volume of the starting sample injected is 0.2-24.0 μl. In some embodiments, the starting sample volume comprises chemical species or a biological species. In some embodiments, the starting sample is partitioned into smaller volumes of 5 pl-5 μl.

These compartmentalized areas allow for isolation of samples and partitioning into a localized array that can subsequently be manipulated and analyzed.

Provided herein is a method for partitioning a sample comprising: a) providing a device comprising a surface, enclosed within a cavity, wherein the surface is selectively partitioned into hydrophobic and/or hydrophilic regions; b) filling the device cavity with a liquid that is immiscible with the starting sample; and c) contacting the starting sample with the device surface such that the sample volume is partitioned in an array defined by the hydrophilic and/or hydrophobic regions across the surface. The surface may be hydrophobic such that the selectively partitioned hydrophobic and/or hydrophilic regions are formed by coating hydrophilic spots on said surface. In some embodiments, the hydrophobic surface is a glass surface, or a black anodized aluminum surface with a thin oxide deposition layer. In some embodiments, the surface is hydrophilic such that the selectively partitioned hydrophobic and/or hydrophilic regions are formed by coating hydrophobic spots on said surface. In some embodiments, the hydrophilic and/or hydrophobic regions are further coated with a binding site for biomolecules selected from a group comprising protein molecules, carbohydrate molecules, nucleic acids and fatty acids. In some embodiments, the surface is selectively partitioned in an array of hydrophilic and/or hydrophobic regions. In some embodiments, the surface is selectively partitioned in hydrophilic and/or hydrophobic regions having dimensions of 5-200 μm and may be selectively partitioned in hydrophilic and/or hydrophobic regions using microfabrication techniques. The microfabrication techniques may include depositions, plasmas, masking steps, transfer printing, screen printing, spotting, spin coating with a lift off (lithography) step, vapor deposition with selective marking, vapor deposition with a lift off (parylene deposition) and pin spotting (dip pen nanolithography) or transfer printing. In some embodiments, the device cavity may be formed by adding a dam structure along the margins of the patterned surface and attaching a cover to the top of the dam. In some embodiments, the cover is a glass lid. In some embodiments, the device cavity has one or more fill ports for loading the starting sample volume into the device cavity which may or may not be included in the cover. In some embodiments, the immiscible liquid is an organic liquid, such as for example a mineral oil such as silicon oil or a fluorinated oil. In some embodiments, the starting sample is partitioned into smaller volumes by contacting it with the selectively partitioned surface by oscillating the device in a to and fro motion or by contacting the surface with the selectively partitioned surface by employing a magnetic force across the surface. In some embodiments, the volume of the starting sample injected is 0.2-24.0 μl. In some embodiments, the starting sample volume comprises chemical species or a biological species. In some embodiments, the starting sample is partitioned into smaller volumes of 5 pl-5 μl.

Some aspects of the technology comprise a method for partitioning a chemical or biological sample volume, into discrete smaller volumes, comprising: a) providing a device for partitioning a sample volume, said device comprising a surface, enclosed within a cavity, selectively patterned into hydrophobic and hydrophilic regions; b) filling the device cavity with a liquid having opposite polarity to that of sample volume; and c) contacting the sample volume with the device surface such that the sample volume is partitioned as per the polarity of the sample in to an array defined by the hydrophilic/hydrophobic patterns across the surface. In some embodiments, the surface is hydrophobic and the selectively partitioned hydrophobic and/or hydrophilic regions are formed by coating hydrophilic spots on said surface. In some embodiments, the hydrophobic surface is a glass surface. In certain other embodiments, the surface is hydrophilic and the selectively partitioned hydrophobic and/or hydrophilic regions are formed by coating hydrophobic spots on said surface.

In some embodiments, the hydrophilic/hydrophobic regions further comprise a coating or deposition of binding agents such as the binding sites for biomocelcules selected form a group comprising protein molecules, carbohydrate molecules, fats and nucleic acids. Some examples of such binding sites may include.

In some embodiments, the said surface is selectively partitioned in an array of hydrophilic and/or hydrophobic regions. In certain other embodiments, the dimensions of the hydrophilic and/or hydrophobic regions are 5-200 μm.

In some embodiments, the microfabrication technique is selected from a group comprising depositions, plasmas, masking steps, transfer printing, screen printing, spotting, spin coating with a lift off (lithography) step, vapor deposition with selective marking, vapor deposition with a lift off (parylene deposition) and pin spotting (dip pen nanolithography). In some embodiments, the microfabrication technique used is transfer printing.

In some embodiments, the device cavity is formed by adding a dam structure along the margins of the patterned surface and attaching a cover to the top of the dam. In some embodiments, the cover is a glass lid. In certain other embodiments, the device cavity has one or more fill ports for loading the starting sample volume into the device cavity. In some embodiments, the fill port is included in the cavity cover.

In some embodiments, the immiscible liquid is an organic liquid. In some embodiments, the organic liquid is a mineral oil. In some embodiments, the mineral oil is selected from a group comprising silicone oil or fluorinated oil.

In some embodiments, the starting sample is partitioned into smaller volumes by contacting it with the selectively partitioned surface by oscillating the device in a to and fro motion. In certain other embodiments, the starting sample is partitioned into smaller volumes by contacting it with the selectively partitioned surface by employing a magnetic force.

In some embodiments, the volume of the starting sample injected is 0.2-24.0 μl and comprises chemical species and/or biological species. In some embodiments, the starting sample further comprises assay reagents. In some embodiments, the starting sample is partitioned into smaller volumes of 5 pl-5 μl. In some embodiments, the starting sample is partitioned into 3,100-3,500,000 smaller volumes.

In some embodiments, the partitioned sample volumes are half volumes shapes on the device surface. In some embodiments, the half volumes are half spheres.

In some embodiments, the device comprising the partitioned sample is compatible for nucleic acid detection and quantitation of nucleic acids. In some embodiments, the step of detecting or determining the amount is performed using a PCR apparatus. In some embodiments, the PCR apparatus is preferably digital PCR.

Other aspects of the technology comprise a device for partitioning a sample comprising: a device cavity filled with a fluid immiscible with the starting sample and a surface, enclosed within the cavity, wherein the surface is selectively partitioned into hydrophobic and/or hydrophilic regions. In some embodiments, the surface is hydrophobic such that the selectively partitioned hydrophobic and/or hydrophilic regions are formed by coating hydrophilic spots on said surface. In certain other embodiments, the surface is hydrophilic and the selectively partitioned hydrophobic and/or hydrophilic regions are formed by coating hydrophobic spots on said surface.

In some embodiments, the hydrophilic/hydrophobic regions further comprise a coating or deposition of binding agents such as the binding sites for biomolecules selected form a group comprising protein molecules, carbohydrate molecules, fats and nucleic acids.

In some embodiments, the said surface is selectively partitioned in an array of hydrophilic and/or hydrophobic regions. In certain other embodiments, the dimensions of the hydrophilic and/or hydrophobic regions are 5-200μ.

In some embodiments, the microfabrication technique is selected from a group comprising depositions, plasmas, masking steps, transfer printing, screen printing, spotting, spin coating with a lift off (lithography) step, vapor deposition with selective marking, vapor deposition with a lift off (parylene deposition) and pin spotting (dip pen nanolithography). In some embodiments, the microfabrication technique used is transfer printing.

In some embodiments, the device cavity is formed by adding a dam structure along the margins of the patterned surface and attaching a cover to the top of the dam. In certain other embodiments, the device cavity has one or more fill ports for loading the starting sample volume into the device cavity. In some embodiments, the fill port is included in the cavity cover.

In some embodiments, the device is further adapted to undergo an oscillatory motion. In certain other embodiments, the device is configured to employ a magnetic force for partitioning the sample. In some embodiments, the device surface is selectively partitioned into 3100-3500000 hydrophobic and/or hydrophilic regions. In some embodiments, the device comprising partitioned sample volumes if compatible nucleic acid amplification in a PCR machine for detection and quantitation. In some embodiments, the PCR machine is a digital PCR.

Another aspect of the technology is a method for performing nucleic acid amplification, wherein the method comprises the following steps a) providing a starting sample containing at least the target nucleic acid; b) providing a device comprising a surface, enclosed within a cavity, wherein the surface is selectively partitioned into hydrophobic and/or hydrophilic regions; c) filling the device cavity with a liquid that is immiscible with the starting sample; d) contacting the starting sample with the device surface such that the sample volume is partitioned in an array defined by the hydrophilic and/or hydrophobic regions across the surface; and e) detecting the nucleic acid strands within the smaller sample volumes.

In another aspect of the technology, the number of spots is predetermined to match the size of the starting sample volume. Further, in certain embodiments, arrays are formed to include known numbers of different diameters for example 200,000 spots of 10 microns each, 200,000 spots of 25 microns each and 200,000 spots of 50 microns each. In some embodiments, the range of partitioned sample volume sizes may be pre-determined and tailored to fit a particular application. In some aspects, the technology further provides methods of processing a plurality of starting samples in parallel.

The simple geometry makes the device easy to use and implement, economical to fabricate and operate, and robust in its operations, that solves the problems associated with currently used systems. The method and device also reduce the number of sample handling steps. Once the sample has been loaded into the plate, no further sample handling steps are required. Furthermore, the patterned surface can be very inexpensive to fabricate. They can be pre-patterned in very high volume using well characterized processes. The device has the added advantage of 100% sample utilization with no dead volume and no sample loss.

### BRIEF DESCRIPTION OF THE DRAWINGS

FIG. 1 depicts one embodiment of the device;

FIG. 2 is a flow diagram showing one embodiment of the method of use of the device provided herein; and

FIG. 3 depicts one embodiment of the device.

### DETAILED DESCRIPTION

The section headings used herein are for organizational purposes only and are not to be construed as limiting the described subject matter in any way. All literature cited in this specification, including but not limited to, patents, patent applications, articles, books, and treatises are expressly incorporated by reference in their entirety for any purpose. While the present teachings are described in conjunction with various embodiments, it is not intended that the present teachings be limited to such embodiments. On the contrary, the present teachings encompass various alternatives, modifications, and equivalents, as will be appreciated by those of skill in the art.

Provided herein is a method for partitioning a sample comprising: a) providing a device comprising a surface, enclosed within a cavity, wherein the surface is selectively partitioned into hydrophobic and/or hydrophilic regions; b) filling the device cavity with a liquid that is immiscible with the starting sample; and c) contacting the starting sample with the device surface such that the sample volume is partitioned in an array defined by the hydrophilic and/or hydrophobic regions across the surface. The surface may be hydrophobic such that the selectively partitioned hydrophobic and/or hydrophilic regions are formed by coating hydrophilic spots on said surface. In some embodiments, the hydrophobic surface is a glass surface, or a black anodized aluminum surface with a thin oxide deposition layer. In some embodiments, the surface is hydrophilic such that the selectively partitioned hydrophobic and/or hydrophilic regions are formed by coating hydrophobic spots on said surface. In some embodiments, the hydrophilic and/or hydrophobic regions are further coated with a binding site for biomolecules selected from a group comprising protein molecules, carbohydrate molecules, nucleic acids and fatty acids. In some embodiments, the surface is selectively partitioned in an array of hydrophilic and/or hydrophobic regions. In some embodiments, the surface is selectively partitioned in hydrophilic and/or hydrophobic regions having dimensions of 5-200 μm and may be selectively partitioned in hydrophilic and/or hydrophobic regions using microfabrication techniques. The microfabrication techniques may include depositions, plasmas, masking steps, transfer printing, screen printing, spotting, spin coating with a lift off (lithography) step, vapor deposition with selective marking, vapor deposition with a lift off (parylene deposition) and pin spotting (dip pen nanolithography) or transfer printing. In some embodiments, the device cavity may be formed by adding a dam structure along the margins of the patterned surface and attaching a cover to the top of the dam. In some embodiments, the cover is a glass lid. In some embodiments, the device cavity has one or more fill ports for loading the starting sample volume into the device cavity which may or may not be included in the cover. In some embodiments, the immiscible liquid is an organic liquid, such as for example a mineral oil such as silicon oil or a fluorinated oil. In some embodiments, the starting sample is partitioned into smaller volumes by contacting it with the selectively partitioned surface by oscillating the device in a to and fro motion or by contacting the surface with the selectively partitioned surface by employing a magnetic force across the surface. In some embodiments, the volume of the starting sample injected is 0.2-24.0 μl. In some embodiments, the starting sample volume comprises chemical species or a biological species. In some embodiments, the starting sample is partitioned into smaller volumes of 5 pl-5 μl.

A device for partitioning a sample comprising: a cavity, a surface located within the cavity, wherein the surface comprises at least one hydrophilic region; and a hydrophobic region surrounding the at least one hydrophilic region.

A method of performing nucleic acid amplification, the method comprising: a) providing a starting sample comprising at least the target nucleic acid; b) providing a device comprising a surface, enclosed within a cavity, wherein the surface is selectively partitioned into hydrophobic and/or hydrophilic regions; c) filling the device cavity with a liquid that is immiscible with the starting sample; d) contacting the starting sample with the device surface such that the sample volume is partitioned in an array defined by the hydrophilic and/or hydrophobic regions across the surface; and e) detecting the nucleic acid strands within the smaller sample volumes.

Provided herein is a device for partitioning of sample volumes into small individual volumes for subsequent assays and analysis. Analysis may involve the analysis of species including but are not limited to, chemicals, biochemicals, genetic materials, or biological cells. The device may be used for applications such as, for example purposes only, polymerase chain reaction (PCR), nucleic acid sequence-based amplification (NASBA), sequencing, crystallization of proteins and small molecules, and the analysis of rare cells or circulating tumor cells present in biological fluids or any other suitable application.

The practice of the present embodiments may employ conventional techniques and descriptions of organic chemistry, polymer technology, molecular biology (including recombinant techniques), cell biology, biochemistry, and immunology, which are within the skill of the art, in light of the present teachings. Some conventional techniques include, but may not be limited to, microfabrication techniques such as depositions, plasmas, masking steps, transfer printing, screen printing, spotting, pin spotting, vapor deposition and spin coating. Specific illustrations of suitable techniques may be described in example herein below. However, other equivalent conventional procedures may also be used.

Nucleic acid detection and quantitation techniques include, but are not limited to, oligonucleotide synthesis, hybridization, extension reactions and detection of hybridization using a label. Specific illustrations of suitable techniques may be described in example herein below. However, other equivalent conventional procedures may also be used. General conventional techniques and their descriptions can be found in standard laboratory manuals such as Genome Analysis: A Laboratory Manual Series (Vols. I-IV), PCR Primer: A Laboratory Manual, and Molecular Cloning: A Laboratory Manual (all from Cold Spring Harbor Laboratory Press, 1989), Gait, “Oligonucleotide Synthesis: A Practical Approach” 1984, IRL Press, London, Nelson and Cox (2000), Lehninger, Principles of Biochemistry 3^{rd} Ed., W. H. Freeman Pub., New York, N.Y. and Berg et al. (2002) Biochemistry, 5^{th} Ed., W. H. Freeman Pub., New York, N.Y. all of which are herein incorporated in their entirety by reference for all purposes.

In some embodiments, a surfactant may be added to the sample to change the amount of surface area of the droplet that is exposed. In some embodiments, the droplet may include the sample and any other necessary and suitable component combined with the sample including, any reagents necessary for PCR including primers and probes, dyes, enzymes, and beads. In some embodiments, the droplet partitioned on the surface of the of the device may be an emulsion comprising other droplets.

### Devices and Methods

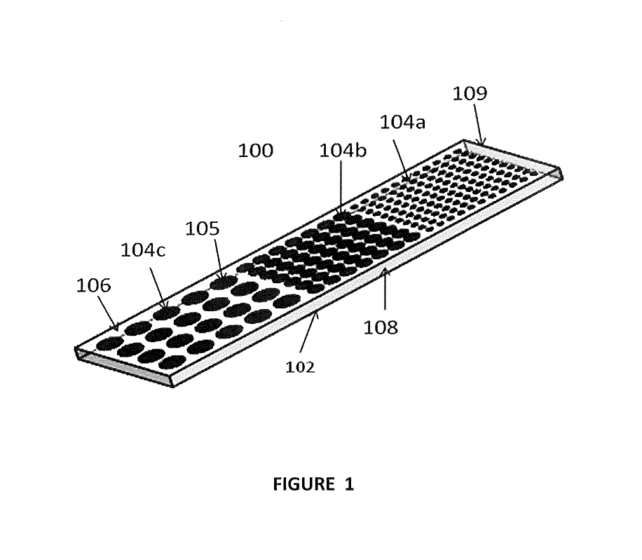

Provided herein is a device for sample volume apportionment and manipulation. One embodiment of the device 100 is shown in FIG. 1. The device 100 may include a substrate 102 or a surface onto which a surface coating may be patterned. In some embodiments, the surface coating may be hydrophilic. In some embodiments, the surface coating maybe hydrophobic. On the surface 102 either hydrophilic or hydrophobic regions 105 may be patterned, which regions 105 are in contrast to the surface coating. The relative hydrophobicities and hydrophilicities of the devices described herein are such as to ensure partitioning of sample volumes across the patterned surface as per the relative polarity of surface and starting volume. The required levels of hydrophobicity and hydrophilicity may vary depending on the nature of the sample, but may be of any suitable level required.

In some embodiments, the substrate or surface 102 may be, glass, metal, silicon, ceramic, composite material, or any other suitable surface onto which a sample may be deposited and then partitioned. In some embodiments, the surface itself may be hydrophobic or hydrophilic. In some embodiments, the surface may be coated with a coating that is hydrophobic or hydrophilic. In some embodiments, the coating may be hydrophobic but, upon exposure to a catalyst, such as current, heat, light, or any suitable form of energy, may become hydrophilic.

As used herein, selectively patterned surface refers to a surface 102 coated with an array of regions/spots 105 of polarity opposite to that of the surface itself. In certain embodiments, a hydrophobic surface is coated with an array of hydrophilic regions/spots, while in certain other embodiments; a hydrophilic surface is coated with an array of hydrophobic regions/spots. The patterned regions 105 may all be of the same size or alternatively, the patterned region may be made of up of regions of varying sizes. In some embodiments, the number and size of the regions may be predetermined based on the size of the starting sample volume. For example, arrays may be formed to include known numbers of different diameters for example 200,000 spots of 10 microns each, 200,000 spots of 25 microns each and 200,000 spots of 50 microns each.

In some embodiments, the regions may be flat regions on a flat surface. In some embodiments, the regions may be bumps or protrusions extending from the top of the substrate to increase the surface area of the spot.

In some embodiments, the patterned regions 105 may be the same shape or they may vary in shapes. For example, the patterned regions may be circular, triangular, rectangular, square, hexagonal, irregular, or any other suitable shape. In some embodiments, the regions are circular in shape and may have a diameter of about 1 to about 250 μm, about 5 to about 200 μm, about 10 to about 200 μm, about 20 to about 200 μm, about 50 to about 200 μm, or about 100 to about 200 μm.

The hydrophilic/hydrophobic regions 105 may be patterned onto the device surface 102 using microfabrication techniques such as deposition, plasma, masking steps, transfer printing, screen printing, spotting, spin coating with a lift off (lithography) step, vapor deposition with selective marking, vapor deposition with a lift off (parylene deposition), pin spotting (dip pen nanolithography), etching, or any other suitable technique.

The regions may further comprise a coating or deposition of binding agents or any other suitable biological molecules. The binding agents may be carbohydrates, sequence specific binding agents, DNA binding agents, or any other suitable binding agent. In some embodiments, a biological molecule or biomolecule may bind to the surface through hydrophobic attachment, electrostatic interactions, covalent immobilization, surface chemistry modification, surface Plasmon resonance, or any other suitable interaction which may cause a biomolecule to bind to a surface. In some embodiments, each region may capture at least one copy of a target nucleic acid.

As further shown in FIG. 1, a sample may be partitioned into discrete smaller volumes on the surface 102. As used herein, “partitioning” refers to breaking down the ‘sample volume” into smaller discrete volumes such that the sum of the smaller volumes is equal to the initial starting sample volume. In some embodiments, the discrete volumes 105 may be the same size or they may vary in size. The discrete volume sizes may be defined by the size of the hydrophilic and/or hydrophobic regions coated on the surface 102. FIG. 1 shows an embodiment of the device with regions 105 of different areas (104a, 104b, 104c) and/or volumes. In certain embodiments, the discrete volumes 105 may be the same shape or they may vary in shape. The patterned region 105 may be circular, square, ovoid, star-shaped, rectangular or any other suitable shape. In some embodiments, the surface of the substrate may be divided into regions. In some embodiments, the surface of the substrate may have regions 105 that are the same size throughout. Alternatively, the substrate may have regions 105 that are divided based on volume. For example, the substrate 102 may have regions 105 with three separate volumes 104a, 104b, 104c, that may be localized to a specific area of the substrate 102 as shown in FIG. 1. Alternatively, the regions 105 of different volumes maybe arranged randomly throughout the surface of the substrate. In some embodiments, the substrate has regions that are of at least one area size. In some embodiments, the substrate has regions that are of at least two different area sizes. In some embodiments, the substrate has regions of at least three different area sizes. In some embodiments, the substrate has regions of at least five different area sizes. In some embodiments, the substrate has up to 10 different area sizes.

In some embodiments, the discrete partitioned samples have volumes of about 5.0 pl to about 5.0 μl, about 50.0 pl to about 5.0 μl, about 500 pl to about 5.0 μl, about 5.0 nl to about 5.0 μl, about 50 nl to about 5.0 μl, about 500 nl to about 5.0 μl, about 1.0 nl to about 2.5 μl. In some embodiments, these volumes may include volumes of fluids or particles to be combined with the sample including, but not limited to, reagents, primer solution, mastermix, magnetic beads, lysing agents, buffers, fluorescent probes, or any other suitable liquid or particle. The number of partitioned volumes on the patterned surface is such that their combined volume is representative of the starting sample volume. In some embodiments, starting volume may be partitioned into a set of smaller volumes, each set having about 3000 to about 3500000 droplets, e.g., about 100000 to about 3000000, about 10000 to about 1000000, about 5000 to about 100000, about 3000 to about 10000. In other embodiments, at least 10%, 20%, 30%, 40%, 50%, 60%, 70%, 80%, or 90% of the starting sample, or the entire starting sample, is segmented into droplets. The starting sample volumes may vary, and may be, for example about 0.2 to about 24.0 μl, about 0.5 to about 24.0 μl, about 1 to about 24.0 μl, about 5.0 to about 24.0 μl, or about 10.0 to about 24.0 μl.

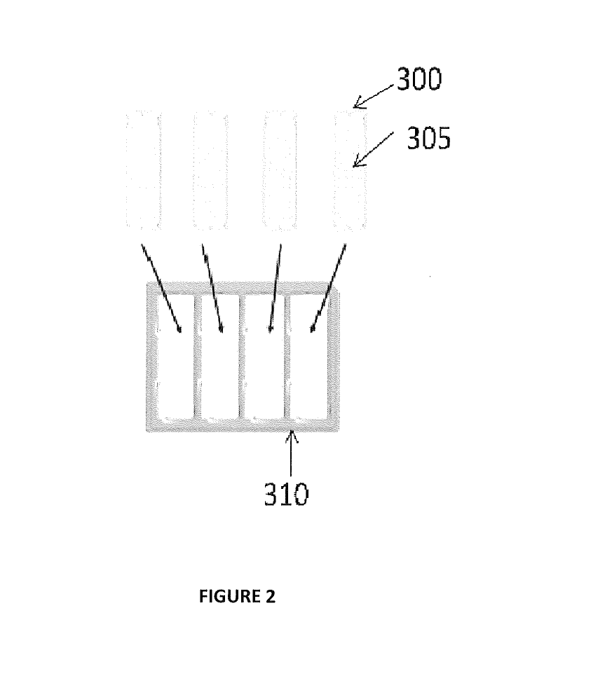

In some embodiments, the sample regions are covered, as seen in FIG. 1. As shown in FIG. 1, the device cavity 108 is formed by adding a dam structure 106 along the margins of the patterned surface 102 and attaching a cover 109 to the top of the dam. The resulting cavity 108 encloses the patterned surface 102. In certain embodiments, the cover 109 may be a glass slip, or any other suitable material. The interior of the cavity 108 may be accessible through one or more openings or ports 207(as further shown in FIG. 2). The openings may be suitable for loading the starting sample volume into the device cavity 108. In some embodiments, the at least one opening or port is included in the cavity cover 109. In some embodiments, the opening is included in at least one of the dam structures located around the periphery of the surface. The cover may be attached to the substrate by any suitable means including but not limited to an adhesive, epoxy, glue, screws, mechanical sealing, thermal sealing, or any other suitable sealing mechanism.

In some embodiments, the device cavity 108 may be filled with a fluid that may be immiscible with the starting sample. In some embodiments, the immiscible fluid may be an oil, such as silicon oil, fluorinated oil, or another other suitable immiscible fluid. In some embodiments, the fluid may be filled in the device cavity before attaching the cover 109. In some embodiments, the fluid may be filled after attaching the cover using the fill port 207.

In some embodiments, the device may be configured and adapted to undergo oscillatory motion by an oscillator, which oscillator may move the device 100 and/or agitate, emulsify, and/or mix the sample. In some embodiments, the device may be configured and adapted to be used in conjunction with a magnetic force.

In some embodiments, the device 100 may be suitable for performing PCR, such as qPCR or digital PCR, or may be suitable for performing sequencing. In some embodiments multiple devices 300 may be processed simultaneously as shown in FIG. 2. Each device 300 may have patterned regions 305 on the surface, which may be of similar patterns or may be of varying patterns. In some embodiments, the devices may have the same patterning or may vary with respect to each other. In some embodiments, the device 300 may be placed in a device holder 310. The device holder may have openings into which the device may be inserted. In some embodiments, the device holder is preloaded with the device and then the sample is then loaded into the individual devices. In some embodiments, the device holder 310 may be adapted and configured to be used with a thermal cycler. In some embodiments, at least two devices, at least three devices, at least four devices, at least six devices may be processed in parallel. In some embodiments, the devices 300 may be processed in series.

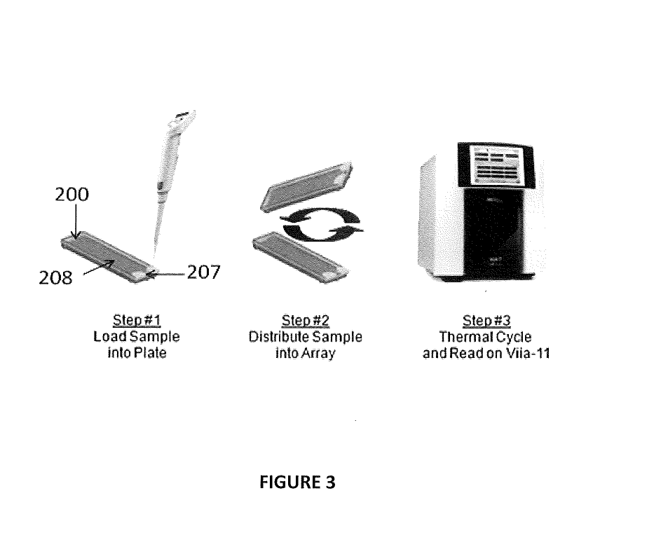

Further provided herein are methods for partitioning a sample volume into smaller discrete volumes. The device 100 of the present invention comprises a device cavity 108 enclosing a surface 102 selectively partitioned into hydrophilic and/or hydrophobic regions 104 which is used for partitioning the starting sample. As shown in FIG. 3, the method includes loading a sample volume 400 in to the device cavity 208 using the fill port 207. In some embodiments, a pipette may be used for introducing the required starting sample into the device cavity as shown in the figure. In certain other embodiments, other suitable means may be employed for loading like using a syringe, dropper, or any other suitable loader for loading the sample. In some embodiments, a vacuum is inside the cavity 208 of the device, which then draws in the sample to load the sample loading.

In some embodiments, the device cavity 208 may first be filled with a fluid immiscible with the starting sample through the fill port 207. In some embodiments, the fluid is an organic oil such as, for example, silicone oil, wherein the starting sample is immiscible in the organic oil. In some embodiments, the fluid is filled before loading the sample. In some embodiments, the fluid is filled after loading the sample into the device cavity. In some embodiments, the device surface may be washed with a suitable solution before loading the sample.

As shown in FIG. 3, after loading the sample, sample is then partitioned into smaller discrete volumes 402 using the hydrophobic and/or hydrophilic properties between the patterned surface and the starting sample. In some embodiments, the device may be shaken or oscillated in order to distribute the sample with the patterned surface. In certain other embodiments, a magnetic force or any other suitable method is employed for partitioning.

The sizes, shapes and numbers of the partitioned volumes may be determined by the patterning of the device surface 102. For example, in some embodiments, the smaller discrete volumes 105 may be the same size or they may vary in size. The discrete volume sizes are determined by the size of the hydrophilic and/or hydrophobic regions 104 coated on the surface 102. For example, in FIG. 1 different sizes of discrete volumes 105 are formed on regions (104) of different diameters (104a, 104b and 104c). In certain embodiments, the discrete volumes 105 may be the same shape or they may vary in shapes defined by the shapes of the patterned regions 104. For example, when the regions are circular they may be half spherical in shape. In some embodiments, the range of droplet sizes may be pre-determined and tailored to fit a particular application. For example, in some embodiments the half spherical droplet sizes and volumes are pre-determined for use in a dPCR apparatus.

After partitioning the sample, the sample is then processed 404. In some embodiments, the processing step may include the use of a thermal cycler as shown in FIG. 3, to prepare the partitioned sample volumes for nucleic acid amplification. In some embodiments, the nucleic acid amplification is carried out using a PCR machine, preferably a digital PCR. In some embodiments, the method may be performed more than once in parallel or in series for simultaneous or successive processing of more than one starting samples. The samples may or may not be identical. In step 1 300, the sample is loaded onto the sample plate. In some embodiments, a pipette or dropper or any other suitable device may be used to dispense the sample onto the device.

Further provided herein is a method of nucleic acid amplification, detection and quantitation. The method comprises of the following steps—a) providing a starting sample comprising at least the target nucleic acid; b) providing device 100 for partitioning the starting sample; c) filling the device cavity 108 with a liquid that is immiscible with the starting sample; d) contacting the starting sample with the device surface 102 such that the sample volume is partitioned in an array defined by the hydrophilic and/or hydrophobic regions 104 across the surface; and e) detecting the nucleic acid strands within the smaller sample volumes.

### EXAMPLES

### Consumable Construction

A standard microscope slide may be used as a platform for performing digital PCR. An array of hydrophilic regions defined as islands on a hydrophobic surface can be formed on the slide using a wide range of microfabrication techniques that are well understood and which have been fully characterized for other applications. These techniques include but are not limited to various depositions, plasmas, masking steps, transfer printing, screen printing and spotting. For the demonstration of concept here the technique of transfer printing was used (see the attachment with an array of droplets). Product requirements and customer needs in conjunction with product cost targets will dictate which process is the most appropriate for manufacturing digital PCR slides.

A grid of hydrophilic regions is defined on the glass slide. A dam structure is formed around the margin of the slide. A glass lid is attached to the top of the dam resulting in an enclosed rectangular cavity on the slide that encloses the hydrophilic array. A fill port is included in the lid. The cavity is filled with silicone oil. A volume of aqueous sample is loaded into the oil filled cavity through the fill port. The aqueous sample falls to the bottom surface of the cavity with the hydrophilic array and the slide is then tilted back and forth. The tilting motion causes the sample droplet to traverse across the array of hydrophilic spots. Each time the sample crosses over a hydrophilic spot some of the aqueous sample material is retained. Tilting the slide continues until the aqueous sample has been transferred to the array of hydrophilic spots. The result is a self aligned array of droplets that are now ready to undergo PCR.

Defining an array of hydrophilic sites has certain advantages. The number of spots can be predetermined to match the size of the sample to be input. Further it is straightforward to form the array of sites to include known numbers of different diameters for example 200 K at 10 microns, 200 K at 25 microns and 200 K at 50 microns. In effect this becomes a parametric emulsion of droplets which provides for great dynamic range for the assay. Of course the range of droplet sizes can be pre-determined and tailored to fit a particular application.

Thermal cycling can be performed on an existing LT flat block thermal cycle and detect platform. This would extend the useful range of those products and eliminate the need to develop a novel platform to support this approach to digital PCR. It would also reduce time to market for this new product as no new platform would be required.

Detection on the same existing platform would be anticipated as well and the same benefits would accrue; however, new detection platforms can be anticipated. One such novel platform would consist of using a CCD chip (or set of CCD chips) under the microscope slide. The array of wells on the slide could be arrayed and aligned to the pixel elements on the CCD chip to optimize sensitivity. The slide reader would image and read the entire population of sites without the need for additional handling of the now amplified sample. Less handling reduces the potential for sample loss. It could be anticipated that a narrow band pass filter or filters would be fitted between the slide with the sample and the CCD chip such that the excitation wavelength could be flooded onto the sample from above and only the emission wavelength would be detected at the CCD chip under the array.

Basic Concept:

A suitably sized plate of either glass, such as a microscope slide, or metal that is patterned with an array of hydrophilic spots that are surrounded by a hydrophobic surface.

An aqueous sample is applied to the surface of the glass plate and the sample is partitioned by the array of hydrophilic capture points.

Without the need for further sample handling and transfer steps, thermal cycling and optical detection is performed and a digital PCR result is obtained.

Anticipated Advantages:

Once the sample has been loaded into the plate, no further sample handling steps are required. Plates, either glass or metal, can be very inexpensive to fabricate. They can be pre-patterned in very high volume using well characterized processes. Sample utilization is 100%; There is no dead volume and no sample loss. Hydrophilic arrays can be custom designed to meet customer requirements. Potential to utilize existing Life Technologies platforms such as Cayenne and ViiA-11 for sample handling followed by thermal cycling and detection would reduce time to begin capturing market share. Features of the array which may be controlled or manipulated include the total number of droplets on plate, individual size of each droplets, pre-defined droplet sets with different sizes to increase dynamic range.

Different loading volumes may be used with the device provided for herein depending on the spot size. Table 1 below shows examples of different loading volumes that may be required depending on the spot size and other variable parameters. Table 1 shows droplet volume (1/2 sphere) droplet size, droplet pitch and the resulting number of droplets on the slide and the minimum volume of sample required fully populate the available spots (hydrophilic attachment sites).

TABLE 1

| | | | | Spots per Plate | |
|---|---|---|---|---|---|
| Spot | Full Spherical | Reaction Volume | Pitch | (20 mm × 70 | Loading Volume |
| Diameter | Volume (uL) | of the Spot (uL) | (um) | mm) | Required (uL) |
| 10 | 5.26E−07 | 2.62E−07 | 20 | 3500000 | 0.92 |
| | | | 30 | 1555556 | 0.41 |
| | | | 40 | 875000 | 0.23 |
| 25 | 8.18E−06 | 4.09E−06 | 50 | 560000 | 2.29 |
| | | | 60 | 388889 | 1.59 |
| | | | 70 | 285714 | 1.17 |
| 50 | 6.54E−05 | 3.27E−05 | 75 | 248889 | 8.14 |
| | | | 85 | 193772 | 6.34 |
| | | | 95 | 155125 | 5.08 |
| 100 | 5.24E−04 | 2.62E−04 | 125 | 89600 | 23.46 |
| | | | 135 | 76818 | 20.11 |
| | | | 145 | 66587 | 17.43 |

The device provided herein may be embodied in other specific forms besides and beyond those described herein. the foregoing embodiments are therefore to be considered in all respects illustrative rather than limiting, and the scope of the invention is defined and limited only by the appended claims and their equivalents rather than by the foregoing description.

Further provided herein are methods of use of the device described herein. In some embodiments the methods may include the steps of: Generally, the methods of the invention include at least the following steps: a) providing a device for partitioning a sample volume, said device comprising a surface, enclosed within a cavity, selectively partitioned into hydrophobic and hydrophilic regions; b) filling the device cavity with a liquid having opposite polarity to that of sample volume; and c) contacting the sample volume with the device surface such that the sample volume is partitioned as per the polarity of the sample in to an array defined by the hydrophilic/hydrophobic patterns across the surface.

What is claimed is:

1. A method for partitioning a sample comprising: a) providing a device comprising a surface, enclosed within a cavity, wherein the surface is selectively partitioned into hydrophobic and/or hydrophilic regions; b) filling the device cavity with a liquid that is immiscible with the starting sample; and c) contacting the starting sample with the device surface such that the sample volume is partitioned in an array defined by the hydrophilic and/or hydrophobic regions across the surface.

2. The method of claim 1, wherein the surface is hydrophobic such that the selectively partitioned hydrophobic and/or hydrophilic regions are formed by coating hydrophilic spots on said surface.

3. The method of claim 2, wherein the hydrophobic surface is a glass surface.

4. The biocompatible system of claim 3, wherein the hydrophobic surface is a black anodized aluminum surface with a thin oxide deposition layer.

5. The method of claim 1, wherein the surface is hydrophilic such that the selectively partitioned hydrophobic and/or hydrophilic regions are formed by coating hydrophobic spots on said surface.

6. The biocompatible system of claim 1, wherein the hydrophilic and/or hydrophobic regions are further coated with a binding site for biomolecules selected from a group comprising protein molecules, carbohydrate molecules, nucleic acids and fatty acids.

7. The method of claim 1, wherein the said surface is selectively partitioned in an array of hydrophilic and/or hydrophobic regions.

8. The method of claim 1, wherein the said surface is selectively partitioned in hydrophilic and/or hydrophobic regions having dimensions of 5-200μ.

9. The method of claim 1, wherein the said surface is selectively partitioned in hydrophilic and/or hydrophobic regions using microfabrication techniques.

10. The method of claim 9, wherein the microfabrication technique is selected from a group comprising depositions, plasmas, masking steps, transfer printing, screen printing, spotting, spin coating with a lift off (lithography) step, vapor deposition with selective marking, vapor deposition with a lift off (parylene deposition) and pin spotting (dip pen nanolithography).

11. The biocompatible system of claim 14, wherein the microfabrication technique used is transfer printing.

12. The method of claim 1, wherein the device cavity is formed by adding a dam structure along the margins of the patterned surface and attaching a cover to the top of the dam.

13. The method of claim 12, wherein the cover is a glass lid.

14. The method of claim 1, wherein the device cavity has one or more fill ports for loading the starting sample volume into the device cavity.

15. The method of claim 14, wherein the fill port is included in the cavity cover.

16. The method of claim 1, wherein the immiscible liquid is an organic liquid.

17. The method of claim 16, wherein the organic liquid is a mineral oil.

18. The method of claim 1, wherein the starting sample is partitioned into smaller volumes by contacting it with the selectively partitioned surface by oscillating the device in a to and fro motion.

19. The method of claim 1, wherein the starting sample is partitioned into smaller volumes by contacting it with the selectively partitioned surface by employing a magnetic force across the surface.

20. The method of claim 1, wherein the volume of the starting sample injected is 0.2-24.0 μl.

21. The method of claim 1, wherein the starting sample volume comprises chemical species.

22. The method of claim 1, wherein the starting sample volume comprises biological species.

23. The method of claim 1, wherein the starting sample is partitioned into smaller volumes of 5 μl-5 μl.

24. A device for partitioning a sample comprising:
a cavity;
a surface located within the cavity, wherein the surface comprises
at least one hydrophilic region; and
a hydrophobic coating covering the surface except for the at least one hydrophilioc region.

25. A method of performing nucleic acid amplification, the method comprising: a) providing a starting sample comprising at least the target nucleic acid; b) providing a device comprising a surface, enclosed within a cavity, wherein the surface is selectively partitioned into hydrophobic and/or hydrophilic regions; c) filling the device cavity with a liquid that is immiscible with the starting sample; d) contacting the starting sample with the device surface such that the sample volume is partitioned in an array defined by the hydrophilic and/or hydrophobic regions across the surface; and e) detecting the nucleic acid strands within the smaller sample volumes.
